(19) United States
(12) Patent Application Publication
(10) Pub. No.: US 2026/0209838 A1
(43) Pub. Date: Jul. 23, 2026

(54) NANOPORE SYSTEM USING SHORT IDENTIFICATION MOLECULES

(71) Applicant: IMEC VZW, Leuven (BE)

(72) Inventors: Mark VEUGELERS, Merelbeke (BE); Koen MARTENS, Aalter (BE); Pol VAN DORPE, Spalbeek (BE)

(21) Appl. No.: 19/141,153

(22) PCT Filed: Dec. 19, 2023
(86) PCT No.: PCT/EP2023/086550

§ 371 Date: Jun. 19, 2025

(30) Foreign Application Priority Data
Dec. 21, 2022 (EP) 22215415.5

(51) Int. Cl. C12Q 1/6869

(52) U.S. Cl. CPC C12Q 1/6869

(57) ABSTRACT

The current disclosure relates to new identification molecules for use in nanopore sensing, said identification molecules having a short backbone comprising densely spaced modifications, and systems thereof, and methods for detecting a target molecule in a sample.

### TECHNICAL FIELD

The present disclosure relates to molecule sensing using nanopores. More specifically, the proposed techniques relate to new identification molecules for use in nanopore sensing, wherein said identification molecules are short and have densely spaced backbone modifications. The disclosure also relates to a system for reading of the new identification molecules, said systems comprising field-effect transistor (FET) nanopores.

### BACKGROUND

Nanopore sensing has been used for DNA and RNA detection and sequencing for some time, and the interest of the research community has now also turned to nanopore sensing of biomolecules such as proteins and peptides. For developing new therapeutic and diagnostic strategies, the possibility to selectively screen a range of proteins at the single-molecule level in biological fluids is a key feature. Nanopore sensing is attractive as it has the potential to detect multiple distinct types of biomolecules, such as DNA, RNA, and proteins, simultaneously in a complex biological sample, and since it can be integrated into small and portable devices.

One method for detecting the presence of analytes in a sample is based on engineering a DNA molecule with structural modifications comprising dumbbells, coupling it to an antibody able to bind a target, incubating it with a target to form a complex with the target, which complex may be translocated through a solid-state nanopore (N. A. W. Bell et al. Nature nanotechnology 11 (2016): 645-652). The engineered DNA molecules may thus function as “barcodes” where the structural modifications give rise to a unique signal pattern when translocating through a nanopore sensor. These barcodes may identify the complex and thus the analyte.

However, the engineered DNA molecules acting as barcodes have several drawbacks, such as folding and forming structures where parts of the modifications are shielded from being detected in the nanopore, and may thus not be read properly. Accordingly, enhanced methods for detecting and quantifying molecules using nanopore sensing are needed.

### SUMMARY

An object of the present disclosure is to provide new identification molecules, methods and systems, which provide alternative and more efficient ways of detecting and quantifying molecules using nanopore sensing, having advantages, such as improved accuracy, low cost and high throughput.

This objective is reached by providing an identification molecule for use in nanopore sensing, wherein the identification molecule is able to generate a unique identification signal when translocated through a nanopore sensor, the identification molecule comprising a backbone molecule having a plurality of backbone modifications, wherein each backbone modification gives rise to a signal magnitude modulation when translocated through the nanopore sensor, and wherein the distance between the backbone modifications of the identification molecule is between 10-40 nt, such as 30 nt, 20 nt or 15 nt, corresponding to 3.4-13.6 nm, such as 10.2 nm, 6.8 nm or 5.1 nm. The densely spaced modifications allow for the use of shorter identification molecules, which have the advantage that folding, and formation of secondary structures, are reduced due to a higher stiffness compared to longer identification molecules.

Hence, in an embodiment, the length of the backbone molecule is 136 nm, or less, corresponding to 400 nucleotides (nt), or less, such as 12-136 nm. In an embodiment, the length is 102 nm, 68 nm, 34 nm, 27.2 nm 20.4 nm 13.6 nm or 6.8 nm, corresponding to 300 nt, 200 nt, 100nt, 80 nt, 60 nt, 40 nt or 20 nt, wherein the length of the backbone is larger than the distance between the backbone modifications.

In the present identification molecule, the backbone modifications are selected from a plurality of modification types, and, upon readout in the nanopore sensor, each signal magnitude modulation attained from the plurality of modifications is assigned an identification value, and wherein all the identification values of the corresponding modifications give rise to an array of identification values corresponding to the unique identification signal of the identification molecule. The identification values are assigned based on i) a distance between the modifications, or ii) the modification type.

In some embodiments, two modification types are used, and each signal magnitude modulation of the two modification types is assigned an identification value of “0” or “1”, and all the identification values of the corresponding modifications give rise to an array of binary identification values corresponding to the unique identification signal of the identification molecule.

In preferred embodiments, the identification molecule has a biopolymer backbone, such as a DNA molecule backbone, preferably a double stranded DNA molecule. The backbone modifications are selected from bulky DNA structures, such as DNA hairpin structures, cruciform DNA, DNA-origami, and quadruplex DNA, DNA-modifications, such as biotin, oligo, peptide, PNA, saccharide, or organic molecules, and differentiated DNA structures, such as ssDNA differentiated into ssDNA hybridized with oligos or poly-ethylene glycol moieties, or a combination thereof. It is also possible to use single stranded DNA as a backbone whereby oligoes of complementary DNA which bind to the single stranded DNA are used as backbone modifications.

In preferred embodiments, the nanopore sensor has a field effect transistor (FET) embedded in the nanopore, such that the FET is gated by an electrolyte-filled nanopore running through the channel region of the FET. In an alternative embodiment, the nanopore sensor comprises a remote extended FET, where an electrode wrapped around the nanopore is connected to a remote gate sensor.

Further is provided a system for target molecule sensing and sequencing using a nanopore sensor, the system comprising: a first electrolyte reservoir and a second electrolyte reservoir, the first and second reservoirs being separated by a barrier comprising one or more FET embedded nanopore comprising an aperture of a predetermined height and a predetermined diameter, and optionally, electrodes for translocating molecules through the nanopore from the first electrolyte reservoir to the second electrolyte reservoir, wherein at least one of the first and second electrolyte reservoirs comprises identification molecules of the present disclosure, wherein the identification molecules are able to generate a unique identification signal when translocated through the nanopore. The identification molecules may be in a complex or optionally separated from the target-identification molecule or target-assay-identification molecule.

The aperture is the narrowest constriction of the nanopore device with which the identification molecule is read. A narrow diameter and low height of said aperture give rise to a strong signal when the identification molecule translocates through the nanopore. The aperture also determines the resolution of the nanopore device, which is the shortest intermodification distance at which the nanopore device can still discriminate the different modifications on the backbone.

In an embodiment, the aperture height h of the FET embedded nanopore is predetermined based on the distance between the backbone modifications of the identification molecule, such that a shorter distance between the backbone modifications determines a shorter aperture height. The aperture height may be in the range of 3.4-14 nm, preferably 7 nm, or less. This corresponds to a spacing between the modifications of approximately 10-40 nt, preferably 20 nt or less.

The aperture diameter is in the range of 3-20 nm, preferably 10 nm, or less. The nanopore FET preferably has a bandwidth of 1 MHz or higher.

The system may be used for carrying out a method of detecting one or more target molecules in a sample using a nanopore sensor, the method comprising: providing (S1) a sample comprising one or more target molecules to be detected; providing (S2) at least one identification molecule according to the present disclosure for the respective target molecules, wherein the identification molecule is able to generate a unique identification signal when translocated through a nanopore sensor and a correlation between the unique identification signal of the identification molecule and the bound target molecule is known; optionally linking (S3) the at least one identification molecule to an assay molecule; incubating (S4) the at least one identification molecule with a sample potentially comprising one or more target molecules, thereby allowing the at least one identification molecule to bind to the one or more target molecules forming at least one target-identification molecule or target-assay-identification molecule, translocating (S5) the identification molecule through a nanopore sensor to obtain at least one unique identification signal, wherein the identification molecule is in a complex or optionally separated from the target-identification molecule or target-assay-identification molecule; and detecting (S6) any presence of the one or more target molecules in a sample by correlating the obtained at least one unique identification signal to the one or more target molecules.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

The above, as well as additional objects, features, and advantages of the present inventive concept, will be better understood through the following illustrative and non-limiting detailed description of different embodiments of the present inventive concept, with reference to the appended drawings, wherein:

FIG. 1A is an illustration of using two different types of side modifications to give rise to a nanopore readout of peaks, being assigned a unique identification signal/barcode. FIG. 1B illustrates a molecule and its resulting signal readout.

FIG. 2A illustrates a backbone with side chain modifications of different types, and FIG. 2B illustrates a backbone with side modifications passing through a nanopore sensor giving rise to a peak pattern, and FIG. 2C illustrates that a backbone with modifications give rise to a peak pattern that may be translated into values, which may be represented by zeroes and ones.

FIG. 3 illustrates a dsDNA backbone having modifications thereon passing through a nanopore, where FIG. 3A is a generic representation and FIG. 3B shows a nanopore FET.

FIG. 4A illustrates barcodes linked to a nucleic acid assay molecule enabling detection of nucleic acid target molecules, and FIG. 4B illustrates barcodes linked to an antibody assay molecule enabling detection of protein antigen.

FIG. 5A illustrates different side modifications on a backbone forming an identification molecule, FIG. 5B shows the resulting read out from the identification molecule in 5A, and

FIG. 6 illustrates different approaches for production of identification molecules, nucleotide oligos upon which modifications, e.g. perpendicular DNA extensions leading to branched oligo's, are placed, whereby different branches may have different lengths, leading to different sized peaks upon nanopore translocation.

FIG. 7 illustrates a DNA ligation based approach for synthesizing the identification molecules.

FIG. 8 illustrates an identification molecule of the present disclosure.

FIG. 9 is a cross-section schematic of a nanopore FET device.

### DETAILED DESCRIPTION

Aspects of the present disclosure will be described more fully hereinafter with reference to the accompanying drawings. The system and methods disclosed herein can, however, be realized in many different forms and should not be construed as being limited to the aspects set forth herein. Like numbers in the drawings refer to like elements throughout. For clarity and simplicity, the method will be described in terms of ‘steps’. It is emphasized that steps are not necessarily processes that are delimited in time or separate from each other, and more than one ‘step’ may be performed at the same time in a parallel fashion.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

In some embodiments a non-limiting term “molecule” as in “target molecule” is used. This refers to any type of analyte that may be detected, identified, and quantified using the methods and system of the invention.

In some embodiments a non-limiting term “biomolecule” is used. The term “biopolymer” may also be used interchangeably. The term “target biomolecule” or “target biopolymer” may be used, and thus refers to the biomolecule/biopolymer that the methods of the invention tries to detect. The target biopolymers or biomolecules herein can be any type of biopolymers, such as polynucleotides, polypeptides, lipids, or polysaccharides, including DNA- or RNA-polymers, peptides, and proteins.

In some embodiments, the generic terminology “identification molecule”, is used. The term “barcode molecule” may be used interchangeably when the identification molecule comprises a barcode. An identification molecule or barcode molecule as used herein refers to an engineered elongated molecule that give rise to an identification signal or barcode signal when translocated (read out) on a nanopore sensor. The identification molecule has a “backbone”, which may in some embodiments be referred to as “backbone molecule”, “scaffold” or “scaffold backbone”, such as a biopolymer backbone or a synthetic non-natural polymer backbone, which comprises one or more modifications/backbone modifications, which are added to attain the identification molecule. The backbone molecule is preferably a double stranded (ds) DNA molecule biopolymer backbone. The biopolymer backbone may also be for example double stranded (ds) RNA, peptides, saccharides, and lipids, and the synthetic backbones may be for example polyesters, polyamides, rigid-rod polymers and/or modified polymers with carbon linker segments. The backbone may also be an uncharged molecule, for example polyethers, such as polyethylene glycol, PEG, or variants thereof.

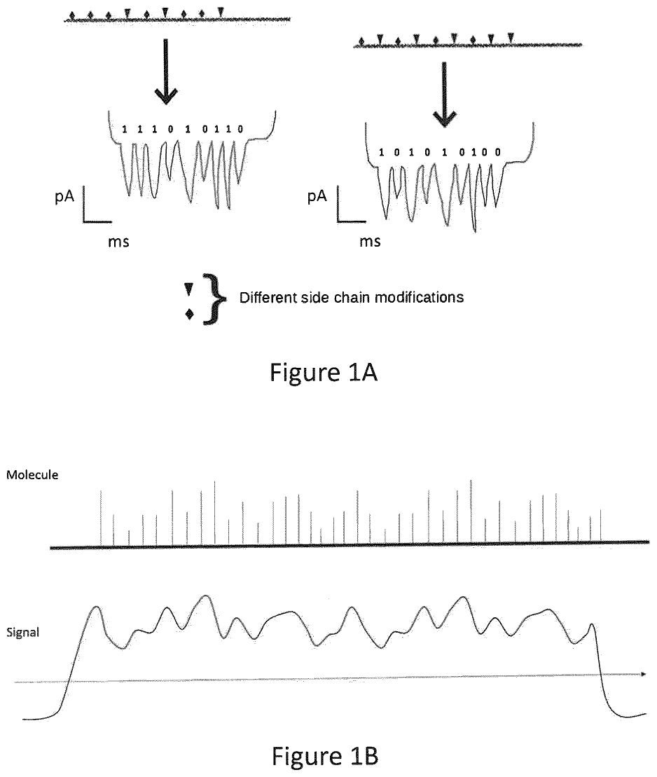

The term “modification” or “backbone modification” refers to a modification on the backbone of the identification molecule, which modifications give rise to the unique identification signal or signal change, or signal alternation, when read out in a nanopore sensor. In some aspects a modification on the backbone, e.g. to the backbone itself, is configured to create a unique signal pattern, a signal magnitude modulation, such as a current peak pattern, when the identification molecule is translocated through the nanopore. The characteristics of the signal pattern can be used as a barcode to identify the target molecule. For example, the change in the signal (signal magnitude modulation) may be a “raising” or “dropping” signal, or a change in the characteristics of the signal, such as in duration or amplitude of a “raising” or “dropping” signal (peaks). In some embodiments, the changing signal pattern as a whole can be used as a barcode. In some embodiments, a series of changes can be assigned as a value in the barcode. One example is shown in FIG. 1. FIG. 1A is an illustration of using two different types of side modifications to give rise to a nanopore readout of peaks, being assigned a unique identification signal/barcode. FIG. 1B illustrates a molecule and its resulting signal readout.

In some embodiments, the modification of the backbone may be by adding a protruding modification to the backbone, in which case the modification/backbone modification may also be referred to as a “side modification”, such as a DNA hairpin, which results in a peak signal when the identification molecule is translocated through the nanopore. For example, a DNA structure extension (e.g. hairpin) may be placed at a single nucleotide location and protrudes from the backbone molecule. The modification protrudes from the backbone and may thus be registered by the nanopore sensor, such as a change in current during translocation of the identification molecule. Said modifications may be of different types, referred to as “modification types”, where the modification types may be e.g. different DNA hairpins, such as an 8 base pair (8 bp) long DNA hairpin or a 16 bp DNA hairpin. Each modification give rise to a signal magnitude modulation (e.g. a peak) when the identification molecule translocates through a nanopore sensor. Different modification types may give rise to different magnitudes of the peaks, and/or a different duration of the peak. In some embodiments, two very closely spaced modifications could appear as one longer signal magnitude modulation, not necessarily having two distinguishable peaks.

The signal magnitude modulations (e.g. peaks) attained in the signal when the identification molecule is translocated through the nanopore is for example due to the change in current during translocation of the identification molecule, thus the signal magnitude modulation or “peak” may be a “current peak”, but may also be a “voltage peak” (voltage threshold peak), where the nanopore is a voltage divider in the device.

Each signal magnitude modulation or peak may be assigned an “identification value”, which may also be referred to as “value” only, which value may depend on the magnitude and/or duration of the signal magnitude modulation/peak (i.e. indirectly based on the modification type). These values may be of two or more “types” or “components”, such as “0” and “1” being different components. The exact implementation may depend on several factors such as the resolution of the pore. In the case of DNA hairpins, a big difference in hairpin size, as measured by the length in nucleotides protruding from the backbone, will give different signal magnitude modulation/peak sizes, e.g. a 8 nucleotide (nt) hairpin will give smaller peaks than a 24 nt hairpin and the difference in size may be sufficient to label all 8 nt hairpins as e.g. 0 and all 24 nt hairpins as 1. The difference in peak size between an 8 nt and 9 nt hairpin may be so small that they will be grouped as having the same size and thus same value. The same applies for other types of modifications. In one example, several signal magnitudes, such as 1-5 may be assigned the same identification value, while the signal magnitudes 6-10 may be assigned another value. Also distances between different modifications may be used for assigning a certain identification value, where for example Distance 1 is assigned a value of 0, and Distance 2 is assigned an identification value of 1, for instance, or where for example the presence or absence of modification is assigned a certain identification value. Where the unique signal is a barcode signal, an “identification value” may be referred to as a “barcode value”. In one embodiment, two modification types are used, and the barcode value is one of two components, either a “0” or a “1”. When having more than two modification types, several modification types may give rise to the same value/component. In some aspects, the magnitude of the peak signal in nanopore sensor is used for assigning the identification/barcode value, where for example there are different signal magnitudes for “1” and “0”, and in other aspects the spacing between the peak signals may be used for assigning the identification/barcode value, where for example the presence of a peak is assigned a “1” and no peak is assigned a “0”. All the identification/barcode values of the identification molecule give rise to a pattern, an array of identification/barcode values, which array is referred to as a unique “identification signal” or “barcode signal”. In one embodiment, a plurality of modification types, such as three or more, may be used corresponding to a plurality of identification/barcode values, which give rise to more complex identification/barcode signals.

The backbone molecules of the invention are typically shorter than usual, such as within the range of 6.8-136 nanometres (nm), e.g. 6.8-102 nm, or preferably 6.8-68 nm. Since the distance between two nucleotides in the DNA double helix structure is 0.34 nm, this corresponds to a length of 20-400 nucleotides (nt), e.g. 20-300 nt, or preferably 20-200 nt. This is possible due to a very dense spacing of the backbone modifications, such as the distance between the backbone modifications of the identification molecule being between 10-40 nt, such as 30 nt, 20 nt or 15 nt. This dense spacing allows for more modifications per backbone length, allowing for a shorter backbone of the identification molecule than usual. To accommodate several modifications in one identification molecule, the length of the backbone is always larger than the distance between the backbone modifications.

The identification molecules of the present invention may have a backbone that is encoded as a chain structure, with different sizes and or shapes of “blocks”, such as origami tubes, balls, cubes, or plates. The blocks may have a stable and semi-rigid structure, and may be formed from one or more layers of a nucleic acid, peptide, or protein. The origami structures can be used as backbone to which extensions are added whereby the extensions allow for barcode generation. As an example, one may have “tube-tube-tube-tube” whereby “-” is a linker and whereby a different hairpin extension or another type of side-chain modification is added to each “tube” block, or alternatively one could also make a structure such as a “tube-ball-cube-plate-ball-tube”, whereby “-” is a linker, and whereby each geometric structure or block due to its different size will give a different signal, i.e. the signal from ball will be different from a cube, and also in this way a unique barcode is generated.

In some embodiments, an assay molecule is used for binding the identification molecule to a target molecule. The term “assay molecule” refers to molecules which are designed or intrinsically able to bind target molecules in an assay. This could be any type of bio or synthetic molecule which may be used in an affinity assay of some sort. Each assay molecule is able to specifically bind to a target biomolecule in a sample. The assay molecules may be linked to one or more identification molecules to form an assay-identification molecule. The assay-identification molecule may bind to a target biomolecule when incubated with a sample comprising the target biomolecule, thus forming a target-assay-identification molecule. Assay molecules may be selected from for example proteins, peptides, DNA, RNA, molecularly imprinted polymers, chemical compounds, metal ions, lipids, polysaccharides, vesicles, whole cells, and polystyrene beads. In some embodiments the assay molecule is an antibody, or a binding fragment thereof, such as a single chain variable region fragment, an aptamer or an affimer. The target-assay-identification molecules may be removed from a sample to be analysed, such as by washing any assay-identification molecule that has not bound any target molecule from the sample, and then the full target-assay-identification molecule, or just a separated identification molecule may be translocated through the nanopore, to detect the target.

The nanopore sensor of the present method preferably comprises a solid state nanopore. In preferred embodiments, the nanopore sensor comprises a nanopore field effect transistor (FET). The nanopore sensor may comprise a first and second electrolyte reservoir, respectively, being separated by a barrier comprising a nanopore. The sensor/system may further comprise electrodes for translocating molecules through the nanopore from the first electrolyte reservoir to the second electrolyte reservoir, wherein at least one of the first and second electrolyte reservoirs comprises the separated identification molecules for detecting the target molecule. The nanopore may have an aperture through which the identification molecule is translocated, where the aperture diameter is linked to the approximate diameter of the identification molecules used. Depending on the resolving power of the nanopore, side chain modifications, such as hairpin DNA structures, need to be spaced sufficiently apart to avoid overlapping peaks. Likewise, the resolving power of the nanopore will determine the minimal length the hairpin DNA extension needs to be to give rise to a peak. A nanopore with high resolving power (such as a nanopore-FET) can still resolve peaks using shorter DNA hairpins which are also more closely spaced next to each other. Hence, to realize the use of short identification molecules having densely spaced modifications, the design of the nanopore FET needs to be tuned.

The properties of the nanopore FET need to be adapted for reading the short identification molecules herein. For example, the nanopore FET will need a high bandwidth, such as higher than 1 MHz. The Nanopore FET will need to be designed for a high signal-to-noise ratio (SNR) at high bandwidth for the short identification molecules. Accordingly, the properties of the nanopore FET aperture may be based on the distance between the backbone modifications of the identification molecule. The height, h, of the aperture may for example vary with the distance between the backbone modifications. The aperture should preferably be in the range of 3.4-14 nm, preferably less than 10 nm. The distance or spacing, s, in base pairs (bp) between the modifications may be divided by approximately 2.94 to attain the aperture height, h, in nm, i.e. h=s/2.94. Thus a height of 3.4 nm to 14 nm may be used for a distance (spacing) between the modifications of 10-40 bp (3.4 nm for a spacing of 10 bp, 7 nm for a spacing of 20 bp and 14 nm for a spacing of 40 bp). Also, the diameter, d, of the nanopore aperture needs to be well-controlled, where the diameter should be 20 nm or less, such as preferably 10 nm or less, such as 7 nm or 5 nm. Larger heights and diameters of the aperture will result in decreased signal strength and decreased signal-to-noise ratio. It should be noted that these measurements relate to the pore aperture, and not the pore channel as a whole. By tuning the size of the nanopore to the size of the identification molecule, there is typically less background signal and stronger foreground signal.

In some aspects, the invention relates to a device or system incorporating the nanopore FET, where a nanopore containing device is configured for performing the methods defined above and below. In some aspects, a system or device of the invention comprises several nanopores, which nanopores may perform simultaneous sensing to attain a high throughput in the system.

In some aspects, a system comprising a microfluidic delivery system is provided, where the microfluidic delivery system delivers different molecules, such as a target sample, an identification molecule, an assay molecule, and an assay-identification molecule. The system may also comprise a detection unit, where the detection unit comprises two reservoirs, where the reservoirs are separated by a nanopore. The microfluidic delivery system may be connected with one of the two reservoirs of the detection unit, wherein one of the two reservoirs comprises the identification molecule. The identification molecule may be in a complex or optionally separated from the target-identification molecule or target-assay-identification molecule. Thus, the identification molecule may either be separate/independent, i.e. not bound to any other molecule, in that it may have been previously separated from the other molecules before translocation through the pore, or it may be bound to e.g. the target/complex, such that it is comprised in an entire complex of target-assay-identification molecule, which full complex is to be translocated through the pore. The detection unit may detect one or more unique identification signals obtained from the identification molecules or the entire complex of target-assay-identification molecules as they translocate through the nanopore, and may be configured for detecting any presence of the one or more target molecules in a sample by correlating the obtained at least one unique identification signal to the one or more target molecules. The system may further comprise a control unit, which controls the delivery of the molecules of the microfluidic delivery system, and which controls the detection unit, and provides instructions to the system for performing the methods described above and below, such as incubating the at least one assay-identification molecule with the sample; removing any assay-identification molecule that has not bound any target molecule from the sample; optionally separating the at least one identification molecule from the at least one target-assay-identification molecule; and translocating the target-assay-identification molecule or the separated at least one identification molecule through a nanopore sensor for obtaining the at least one unique identification signal. The incubation step of incubating the at least one assay-identification molecule with the sample may be performed in different location in the microfluidic system, such as pre-mixed in the microfluidic delivery system, as a separate unit or in one of the reservoirs. The step of removing any non-bound assay-identification molecules may employ a washing or removing mechanism configured for washing/removing the any assay-identification molecule that has not bound any target molecule from the sample to a microfluidic output channel. The step of separating the identification molecule from the complex may employ a separation mechanism configured for separating or isolating the at least one identification molecule being separated from the target-assay-identification molecule by photolysis, heat, pH change, chemical removal or enzymatic removal, by, for example, delivering the chemical/enzyme through at least one microfluidic channel or provide heating/pH modulation from at least one electrode or comprising an illumination source, etc. The identification molecule or target-assay-identification molecule may be made to translocate between the two reservoirs through the nanopore for example by applying voltages on at least one electrode located in each reservoir. In the event of no target analyte/molecule present in the sample, this may be detected by the detection unit as the unit detects no changes in the signal corresponding to a barcode signal and brings a negative detection result.

Nanopores are pores of nanometre size that may be created by a pore-forming protein or as a hole in synthetic materials such as silicon or graphene. Nanopores may be organic when formed by pore-forming proteins, or they may be inorganic solid-state nanopores made for example in silicon compound membranes. Nanopores have been shown to function as a single-molecule detector when present in an electrically insulating membrane, where a voltage is applied across the membrane and an ionic current passing through the nanopore is monitored. The membrane, biological or solid-state, comprising the nanopore is surrounded by an electrolyte solution, where the membrane splits the solution into two chambers. An electric field is attained by applying a bias voltage across the membrane, inducing an electric field that drives motion of charged particles (ions). Preferably, all the voltage drop concentrates near and inside the nanopore such that charged particles in the solution only feel a force from the electric field when they are near the pore region, also denoted capture region. Nano-sized polymers, such as DNA or protein having a net charge, will then, if placed in one of the chambers, feel a force from the electric field when near the pore region. Thus, the molecule will be attracted to the pore capture region, enter the nanopore, and inside the nanopore translocate through via a combination of electro-phoretic, electro-osmotic and sometimes thermo-phoretic forces.

For a nanopore of molecular dimensions, passage of molecules, such as DNA, cause interruptions of the current level of the open pore, leading to a signal as the molecule translocates through the pore. Inside the pore the molecule occupies a volume that partially restricts the flow of ions, observed as an ionic current drop. The characteristics of said interruption or disruption of the current may then be used to determine the sequence or identity of the molecule passing through the pore. Based on various factors such as geometry, size and chemical composition, the change in magnitude of the ionic current and the duration of the translocation will vary. Different molecules can then be sensed and potentially be identified based on this modulation in ionic current, which is referred to as nanopore sensing.

Nanopore sequencing is a sequencing technology based on nanopore sensing that may be used for sequencing of DNA and RNA, where a single molecule of DNA or RNA may be sequenced without the need for PCR amplification or chemical labelling. A DNA or RNA sequence may be determined by detecting the bases as the RNA or single stranded DNA (ssDNA) translocates through the pore. Biological nanopores, such as Alpha hemolysin (aHL) and Mycobacterium smegmatis porin A (MspA) exist, and it has been shown that all four bases can be identified using ionic current measured across the aHL pore. Alternatively, solid-state nanopores are used, which do not incorporate proteins into their systems, but instead uses various metal or metal alloy substrates with nanometer sized pores that allow DNA or RNA to pass through. For example, measurement of electron tunnelling through bases as ssDNA translocates through the nanopore is used as a solid state nanopore sequencing method. Alternatively, DNA sequencing using solid state nanopores and fluorescence may be used, where each base is converted into a characteristic representation of multiple nucleotides which bind to a fluorescent probe strand-forming dsDNA, and each base may be identified by two or four separate fluorescences. The biological nanopores being advantageous due to low translocation velocity, i.e. passing slow enough to be measured, and dimensional reproducibility of the pore size. The solid-state nanopores being advantageous due to stress tolerance to environmental conditions, longevity, and ease of fabrication when mass produced. To realize the use of the short identification molecules of the present disclosure, the nanopore sensor has a field effect transistor (FET) embedded in the nanopore, which have specifically adapted aperture measurements.

Even though it has not been previously possible to determine a complete DNA-sequence of an unmodified DNA molecule translocating through a solid-state nanopore from the ion current through the nanopore, it is possible to detect the differential electrical signal of different DNA-structures (e.g. DNA hairpins with different hairpin lengths) and regions with different DNA structure (e.g. ssDNA versus ssDNA hybridized with oligos) with solid-state nanopores and nanochannels. The spacing and type of these DNA-structures can be modified/designed such that the electrical signal readout using a solid state nanopore resembles a barcode.

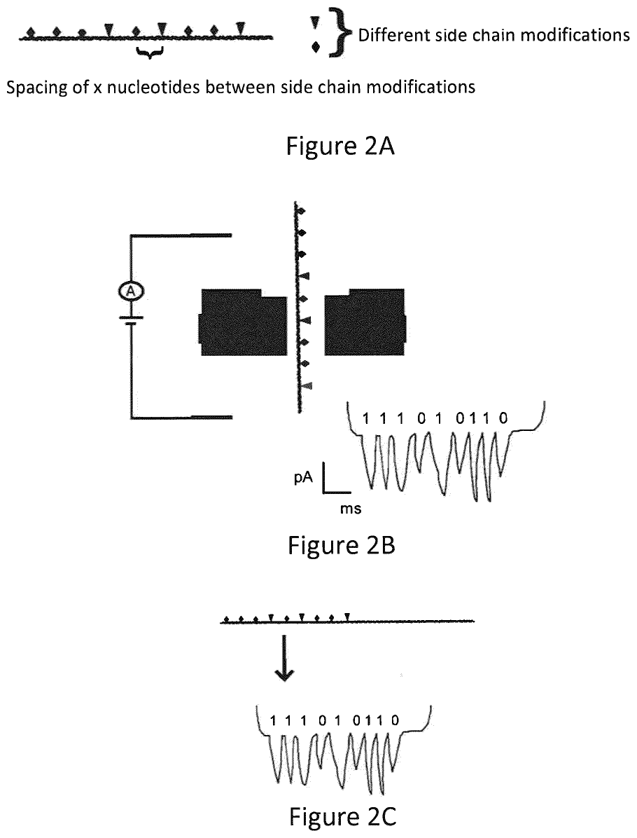

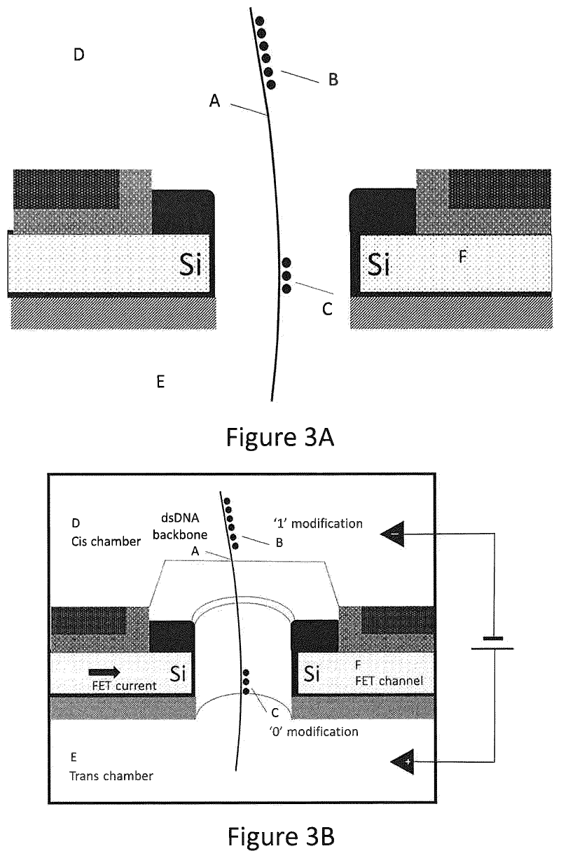

As shown in FIG. 2A, side (chain) modifications, i.e. backbone modifications, may be made to the DNA carrier, which side modifications block the current going through the solid-state nanopore as shown in FIG. 2B, e.g., having DNA stick out from the double strand (e.g. by a dumbbell or hairpin sequence), leading to a peak in the electric signal. By varying the position and type of the dumbbels or hairpins, one can generate a combination of peaks which constitute a barcode pattern, as also shown in FIG. 2B. FIG. 2C also illustrates the relation between side modifications and the attained peak pattern and resulting identification signal 111010110. One such example is illustrated in FIG. 3. FIG. 3A and B illustrates a backbone A, such as a double stranded DNA, having thereon modifications B and C, giving rise to values “1” or “0”, respectively, passing through a nanopore sensor with chambers D and E.

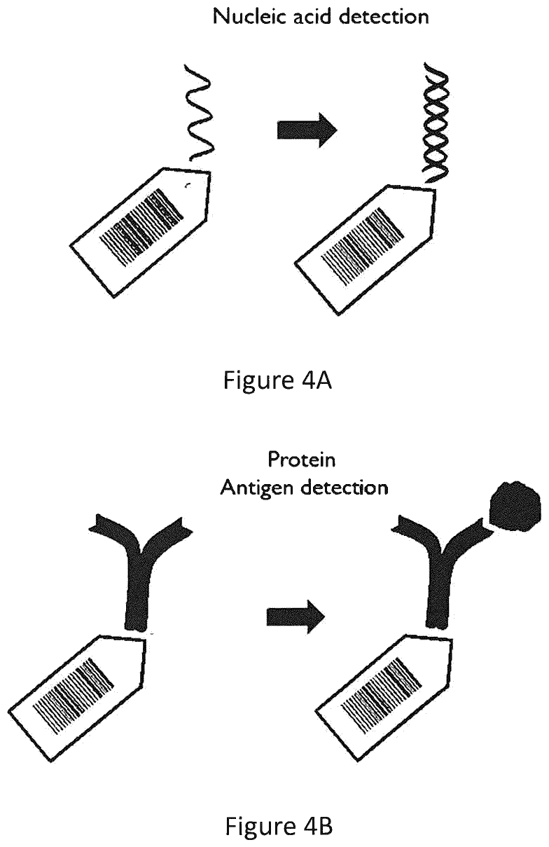

Thus, barcodes may be used both for nucleic acid detection, as well as protein detection, as shown in FIGS. 4A and 4B. A DNA hairpin structure (identification/barcode molecule) may be attached to an RNA capture molecule (assay molecule), allowing hybridisation to a target RNA in a sample, and a photocleavable DNA hairpin structure (identification molecule) may be attached to a protein capture molecule (antibody-assay molecule) allowing binding to the target protein. After photocleavage, the identification molecule is released allowing readout of the barcode in a solid-state nanopore platform. Using a strategy similar to the nCounter technology developed by the company Nanostring, one can perform gene expression analysis by reading out the hairpin barcodes specific for each capture probe. An example of using two different types of modifications (hairpins) with a certain spacing to give rise to a nanopore readout of peaks, which may be assigned certain values (0 or 1), giving rise to a unique signal is illustrated in FIG. 1A.

In order to provide enhanced sensing methods which avoids drawbacks of the prior art, it has been found by the current inventors that by using a nanopore FET with specific measurements, densely spaced backbone modifications of an identification molecule may be read out, and hence the backbone molecule of the identification molecule used may be shorter. This solves the issue when long identification molecules form a bundle, preventing parts of the identification molecule to be read. As shorter identification molecules are stiffer, the problem of non-readable parts of the identification molecule is overcome.

As discussed above, it is possible to detect the differential electrical signal of different modifications (different DNA-structures e.g. DNA hairpins with different hairpin lengths) and regions with different DNA structure (e.g. ssDNA versus ssDNA hybridised with oligos) of an identification molecule (backbone with modifications) with solid-state nanopores and nanochannels. The spacing of these modifications/DNA-structures can be modified/designed such that the electrical signal readout using a solid state nanopore resembles a barcode. However, up to now, these DNA structures have been made using mostly DNA origami approaches and long DNA molecules. It has now been realized by the current inventors that there are several advantages to the use of shorter identification molecules, such as short oligos as a continuous double helix DNA backbone upon which different DNA-structures can be implanted, which result in different electrical signals in a solid-state nanopore, and more specifically a nanopore-FET. The high bandwidth of a nanopore-FET makes it possible to differentiate closely spaced DNA structures, and hence more modifications, being more densely spaced, may fit onto a short backbone than before, whereby these DNA structures result in different electrical signals (signal modifications) upon translocation through the nanopore-FET. A nanopore-FET for use in the present invention will also be able to differentiate a high number of different types of DNA structures, thereby leading to higher barcode potential for the same length of DNA compared to a regular solid-state nanopore.

A method to produce solid state nanopore-readable DNA hairpins has been described in Chen K et al., Nano Lett. 2019 February 13;19(2):1210-1215, where DNA hairpins are used to generate a signal in a nanopore readout which is labelled as a bit (0 or 1) and therefore enables data encoding in DNA-structures. Accordingly, it has been realized that different bits can be used as “barcodes” (identification signals/sequences) for identification, using identification molecules comprising side modifications, e.g. a DNA hairpin structure which gives a solid-state nanopore readout as 1001101110 can in fact be used as a barcode sequence which is different from a DNA-hairpin structure which gives a nanopore readout as 1111101110, hence a different barcode sequence (array/sequence of identification values), which is the unique identification signal.

In the field of molecular biology, DNA-sequence based barcodes, e.g. nucleotide barcodes are frequently used to identify biomolecules. Compared to the identification molecules of the present invention, these DNA-sequence based barcodes are currently not distinguishable by measuring ion currents upon translocation of DNA-sequence based barcodes through solid-state nanopores, while their use in biological, protein-based nanopores is limited due to the current high error rates of DNA sequencing in protein-based nanopores, thus requiring long DNA sequences to take nanopore sequencing errors into account. By transforming a DNA-sequence based barcode into a DNA hairpin structure based barcode, it becomes possible to use solid-state based nanopores to perform the same type of assays as are possible using DNA-sequence based barcodes on current DNA-sequencing platforms. This includes a broad application space such as the use of DNA-encoded chemical libraries, whereby chemical molecules can be tagged with DNA hairpin barcodes.

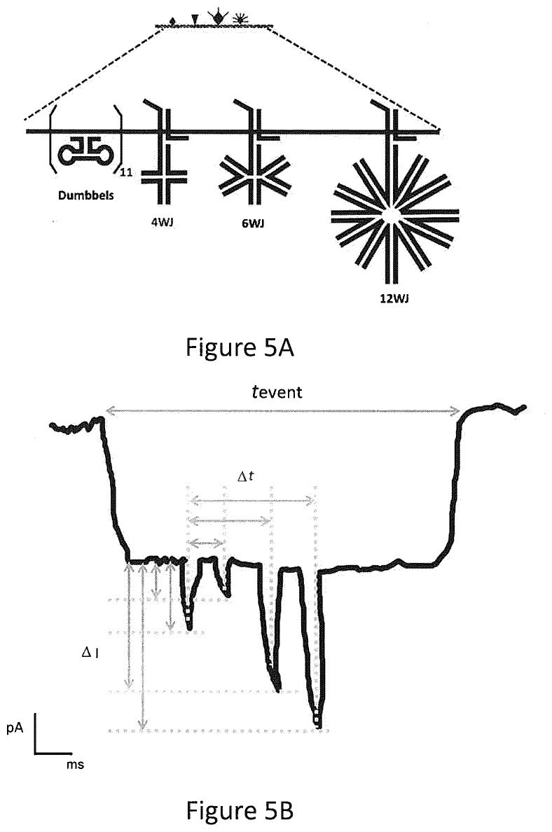

One is not limited to using hairpin DNA sequences as backbone/side modifications of a DNA backbone to design nanopore readable identification molecules/barcodes: any bulky DNA-structure (cruciform DNA, DNA-origami, quadruplex DNA) or DNA-modification (biotin, oligo, peptide, PNA) or different DNA structure (e.g. ssDNA versus ssDNA hybridised with oligos) and combinations of the above can be used to design a solid-state nanopore-readable identification signal, as illustrated in FIG. 5. FIG. 5A shows a schematic view of different side modifications on a backbone forming an identification molecule, including use of dumbbels and three DNA junction structures of different sizes (4-way junction, 6-way junction, and 12-way junction) to generate a quaternary encoding system; other modifications such as DNA-hairpins, 3-way junctions, 8-way junctions etc. are also possible. FIG. 5B shows the resulting read out from the identification molecule in 5A, i.e. nanopore readout showing differentiable signal for each of the 4 types of structures. More than 4 types of nanopore-differentiable encoding systems may be possible depending on the nanopore-FET structure and resolution. A single dumbbell unit is 14 nucleotides long, whereas 11 dumbbell units (220 nucleotides) give a sufficient signal for barcode readout.

Readout of DNA-structure based identification molecules/barcodes on a solid-state nanopore has the advantage compared to DNA sequence based barcode readouts of very low assay cost, and extremely high and fast throughput per assay. Since very long DNA molecules can be translocated through solid-state nanopores, and since it is the distance between DNA hairpins or DNA structures which determines the barcode/array of values of the identification molecule (identification signal/barcode signal), one can use very short identification signals (e.g. 01) in addition to extremely long identification signals (1010110101 . . . ). One can use different identification systems: e.g. the identification signal can be varied in length (e.g. a barcode with 2 components here indicated with 0 and 1, which can give identification signals such as 0011010 or 1110000000), and one can also design complex barcodes whereby multiple components can be used (leading to barcodes of sequence 0a²&, whereby 0, a, ², & represent different DNA-structures.

However, it can be problematic to read out barcode structures when long identification molecules, e.g. long DNA molecules are translocated through a solid-state nanopore. Long identification molecules may form tertiary structures, such as forming a bundle when being translocated, and hence large parts of the identification molecule may be obscured, and hence not read properly. According to some sources, the number of nanopore translocation events excluded due to either incomplete DNA or folded DNA translocating through the nanopore thereby obscuring a correct barcode reading is as high as 85%, see e.g. Bell and Keyser, Nat. Nanotech 2014, Digitally encoded DNA nanostructures for multiplexed, single-molecule protein sensing with nanopores. Hence, this poses a great problem when detecting/sequencing target molecules using such identification molecules.

A solution to this problem could thus be to use shorter identification molecules, which are stiffer and less prone to form bundles. However, this has not been possible using regular solid state nanopores due to the distance needed between the modifications to read out the correct barcode signal. However, by using a nanopore FET (NPFET) with optimized bandwidth and reading aperture measurements, it has now been shown that it is possible to read out modifications being as densely spaced as 10 bp or nt, as compared to the normally used spacing distances, such as 114-1032 bp. This allows for more modifications per backbone length; hence the same number of modifications may fit on a much shorter identification molecule.

A regular solid-state nanopore also has less sensitivity to detect different sized/charged side chains compared to a NPFET nanopore, meaning that use of a NPFET will result in more different types of barcodes per oligo or DNA sequence of the same length detectable compared to a regular solid-state nanopore. Further, the high bandwidth of a nanopore-FET makes it possible to differentiate more closely spaced DNA structures, meaning that shorter DNA sequences can be used than in a regular solid-state nanopore. The combination of these features shows that there may be several advantages to use short oligos (e.g., 200-400 nucleotide oligos) upon which hairpin DNA barcode structures are placed in a nanopore-FET. The advantage of the higher bandwidth offered by a Nanopore-FET means that millions of barcodes could be generated on very short, e.g., 200 nt, oligos. For example, molecular modelling indicates that modifications every 10 nucleotides are detectable with a nanopore-FET. This would mean that 20 binary (or higher) modifications are detectable per 200 nt oligo, leading to at least ~1 million barcodes possible (2²⁰).

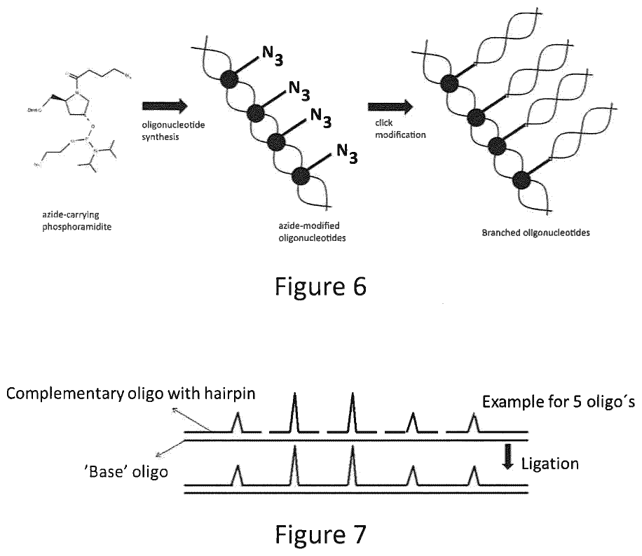

Production of barcoded oligo's can be done using several approaches, as illustrated in FIG. 6. For example, custom azido-nucleotides (for click chemistry based side chain implantation and thus barcode generation) can be incorporated during oligo-synthesis. Smaller oligos can be modified with different side-chain modifications in separate reactions and afterwards ligated together to generate a barcode-oligo. Alternatively, enzymatic modification of the oligo DNA with Taq1 methyltransferase (4 nt recognition sequence) can be performed. A synthetic oligo can be designed with spacing of the recognition sequence every 10 or X nucleotides), where X can vary from e.g. 10 to 40 nt. Different methyltransferases with different target sequence can be used to enable incorporation of different side chains for barcode generation.

While current phosphoramidite chemistry for oligo synthesis suffers from incomplete product formation during production of long oligos with sizes above 100 nt, several strategies can be used to remedy this problem, for example phosphoramidite-produced long oligo's (300 nt) can be purified for size by PAGE or HPLC. Further, one can use commercial synthetic biology based DNA assembly technologies to generate correctly sized oligos. Examples include the BioXP technology from CODEX DNA which uses synthetic biology (Gibson assembly) to assemble short 20-30 nt oligos into longer fragments of correct size. Similar technologies from other companies (e.g. Elegen, Evonetix etc.) are also possible to use.

In one example, a DNA ligation based approach can be used, which may also be referred to as a “Mini origami” approach, as illustrated in FIG. 7. Said approach includes to generate a single ‘base’ oligo of 200 nucleotides, purified for correct length, generate a set of 10 purified complementary oligo molecules (of ~20-30 nt size) which hybridize to the base oligo such that a barcode is generated, ligate fragments together. This approach requires production of 21 purified oligos which than allow production of millions of barcodes using a 0-1 bit encoding system.

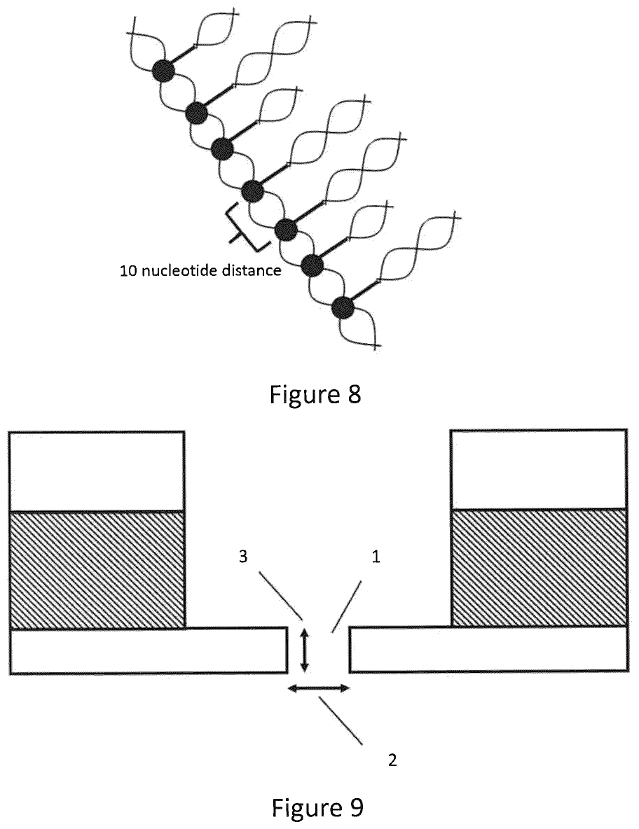

Short oligos with barcode structures placed every 10 nucleotides translocating through the nanopore-FET have several advantages for nanopore sensing. One advantage is higher speed, shorter transit times leading to more barcodes which could be read in the same timeframe, thus leading to higher throughput thereby leveraging the advantages of the NPFET. E.g. a dumbbell structure of 11 units requires a length of 880 nucleotides to generate a barcode structure of 01 or 10 (the 880 nucleotides incl. space for 2 side chain structures to determine orientation upon nanopore translocation). Another advantage is an uninterrupted double helix backbone and stiffer DNA thereby avoiding folding of the backbone and the barcode structure, which reduces errors (DNAs with secondary structure entering the pore result in unreadable barcodes) and lowers noise (less Brownian motion). Another advantage is lower cost (less oligos needed to assemble a barcode). The higher sensitivity and bandwidth of the nanopore-FET will also enable more different types of barcodes per oligo or DNA sequence of the same length detectable compared to a regular solid-state nanopore (more bits/barcode). Further, it reduces molecular weight of barcodes (advantageous for assay reagent kinetics, assay time), for example at ~0.6 kDa/bp, a 2 kbp DNA will be: ~1200 kDa vs. nanobody 12-15 kDa; aptamer 5-15 kDa; antibody: 150 kDa, it avoids origami bending points, no origami folding errors and no folding of backbone, provides less dehybridisation and reduces errors. FIG. 8 illustrates an identification molecule of the present disclosure, having side chain modification present at a distance of 10 nt from each other on the backbone molecule.

In the present disclosure identification molecules may be used for identifying a target in a sample. In some aspects, these identification molecules are designed as nanopore-readable barcodes, where the identification molecules are coupled to assay molecules, such as antibodies, for detecting target biomolecules in a sample, whereby the biomolecules may be indirectly identified and quantified by translocating the identification molecule-assay-target complex, or by separating the identification/barcode molecules from the assay-target complex and translocating the identification molecules only through a nanopore, hence enabling identification of the specific barcode linked to the biomolecule and thus the presence of the biomolecule or a specific part of a biomolecule. By measuring the quantity (number) of translocated identification molecules relating to a specific biomolecule, a quantity or concentration may be determined, such as an absolute concentration or a relative concentration to another measured biomolecule in said sample. The quantification may be made by counting the number of unique signals attained, which may be correlated to the concentration of the target sample. In some aspects, counting of the barcodes indicates the concentration of the target biomolecules as in a “one barcode-one target molecule” scenario.

The structures used as identification molecules (barcode molecules) in the present disclosure need not only be DNA-structures, but may also comprise any type of backbone to which backbone/side modifications are added, such that these modifications result in a barcode signal when read out in a nanopore, preferably a nanopore FET. The backbone may be any linear structure, typically a polymer, which can be used to imprint on it backbone structures (backbone/side modifications) such that upon nanopore translocation, a barcode-like signal, a unique identification signal that comprises an array/sequence of identification values, is observed. Spherical structures (e.g. nanoparticles) or non-linear structures would be limited in their potential to be used for generating a large number of barcodes (unless linked together to form a linear chain), but any type of linear structure would be possible to use if possible to comprise said backbone structures. In some embodiments, the backbone is generated from a charged molecule, such as biopolymers and synthetic polymers. Alternatively, the backbone is generated from an uncharged molecule, for example polyether, such as polyethylene glycol (PEG), or variants thereof. Alternatively, structures such as nanotubes or nanowires can be used. In a preferred embodiment, the backbone molecule is DNA-based, such as ss DNA or ds DNA.

In some embodiments, the backbone is a biopolymer backbone, such as RNA-, peptide-or saccharide biopolymer backbone, to which one or more backbone or side modification is added, such that these modifications result in a barcode signal in a nanopore FET. In other embodiments, the backbone is a synthetic polymer backbone, such as polyesters, polyamides, rigid-rod polymers, and modified polymers with carbon linker segments. In a preferred embodiment, the backbone comprising modifications is a DNA backbone comprising modifications in the form of DNA hairpin structures and DNA dumbbells.

These identification molecules/barcode molecules or side modifications thereof (barcode structures), can then be chemically coupled to another biopolymer such that the biopolymer becomes barcoded, and after assaying and optional removal of the identification molecule, the current signal magnitude modulation, e.g. peak pattern of the identification molecule/barcode structures upon translocation can be read using a the nanopore FET.

Accordingly, the present invention relates to providing, such as generating, short identification molecules comprising a backbone with densely spaced modifications, which, when translocated through a nanopore sensor give rise to an identification signal, said signal either read directly from the identification molecule, or by assigning values to the signal modifications/peaks attained from the modifications of the identification molecule. The backbone or side modifications may be of multiple modification types, which generate different signals/peaks when read out on a nanopore sensor due to the different impacts on the electrical field. Each peak may be assigned an identification value, such as when using two modification types the identification values may be binary barcode values of “0” or “1”, and the complete readout of the identification values of the multiple modifications and corresponding peaks of the identification molecule is referred to as an identification signal, such as 001, 101, or 111 for a three peak readout. Thus, all the identification values of the identification molecule give an array or sequence of identification values in the order of which each peak is attained when the identification molecule translocates through the nanopore, which array/sequence is the identification signal. Accordingly, when using two modification types having identification values of 0 or 1, the identification signal will be a binary signal of zeroes and ones corresponding to the backbone modifications of the identification molecule, where the assignment of identification values for the peaks are predetermined. Thus, each identification molecule comprises a unique identification signal.

The identification molecules may be linked to assay molecules, thereby forming an assay-identification molecule. Assay molecules of the present disclosure are molecules which bind target molecules in an assay, such as an immunoassay, wherein each type of assay molecule is designed to bind a certain target molecule. The assay molecules may be for example DNA, proteins, peptides or polysaccharides, and the assay molecules may be linked to the identification molecule through a variety of methods such as direct conjugation, affinity-based conjugation, hybridization, ionic interaction, or crosslinking, such as crosslinking including click chemistry. Thus, as the assignment of identification values to different peaks is predetermined and accordingly the identification signal of each identification molecule known, and since the linking of a certain (one or more) identification molecule to certain assay molecules binding specific target molecules is also known, one or more identification signals may be correlated to a specific target molecule. Also, when the identification molecule is identified using other methods than intrinsic barcodes, the link between the unique identity of the identification molecule and assay molecule, binding a specific target, is known, and may be used for detecting the target upon identifying the identification molecule in the nanopore sensor.

Thus, to be able to detect certain target molecules in a target sample, one may create a number of identification molecules linked to assay molecules, which each bind a target molecule. The “sample” or “target sample” to be analysed may be a biological sample, such as biological fluids, including blood, serum, plasma, saliva and urine, cells, including cell populations and cell cultures, cell digests, and environmental samples, or a non-biological sample, such as waste water, inorganic molecule solutions, etc., where certain assay molecules (e.g. aptamers) may be used, which recognize metal ions, such that the assay identification molecule could be used to measure the concentration of metal ions in a non-biological sample. By incubating the assay-identification molecules with the target sample, binding of some of the assay-identification molecules to target molecules in the sample will occur. By removing, such as washing away, unbound assay-identification molecules from the sample, only the assay-identification molecules which have bound to target molecules and formed target-assay-identification molecules will remain in the sample. By translocating said complex through the nanopore, or by separating the identification molecule from the target-assay-identification molecule in the sample, such as having a photo cleavable identification molecule and cleaving off the identification molecule using photolysis, where the separation may include isolation of the identification molecules by removing the identification molecule from the sample, the identification molecule may be run through a nanopore sensor, where the different types of backbone/side modifications of the identification molecule give rise to a signal or peak pattern, which peak pattern may be translated into an identification/barcode pattern, e.g. by assigning identification values to the respective peaks, wherein the identification pattern (sequence of identification values) is referred to as an identification signal. In some embodiments, the identification signal is based on the size or translocation time/duration of the identification molecule.

Identification of a specific identification molecule in the nanopore may be correlated to a presence of the target in a sample being used in the assay. In some aspects, the identification molecule in itself may be an identifier, such as the length of it may be correlated to the unique identity. In some aspects, it may carry information, such as modifications, which may be detected in the nanopore as a peak pattern, and assigned identification values, such as barcode values. It should be noted that the “barcode molecules” and “barcode values” of the invention differs from the typical DNA barcode in which the barcode is in the sequence of nucleobases. The unique identity may also be based on transition time between the side modification in the nanopore sensor. If no target molecules are present, then no assay molecules will be bound when incubated with the target sample, and hence no identification molecules will be detected with the nanopore.

The identification molecule can translocate through the nanopore in two directions. For example, if it enters in one direction the signal would be 10001111, the reverse direction will give 11110001. To allow use of a large number of identification molecules, the identification molecule can also be labelled at one of the two ends with a specific signal, thereby establishing directionality.

In some aspects, the identification molecule comprise a small fraction at each end that give rise to a predetermined array of values for detecting the orientation of the identification molecule as it translocates through the nanopore, thereby establishing directionality of the identification molecule as it passes through the nanopore sensor. For example, one may have a “barcode region” specific for orientation purposes in the backbone of the identification molecule. Thus, to allow deduction of the orientation of the molecule translocating through the nanopore, smaller identification molecules or backbone/side modifications may also be attached to the respective sides/ends of the molecule (“left” and “right” side of the molecule, or front/end), said small identification molecules comprising backbone/side modifications which translates into an identification pattern (identification signal) of e.g. 10 modifications each, and where the pattern/signal differs between the “right” and “left” side, and also such that it does not occur in the main identification signal of the target molecule to be detected.

In preferred embodiments, the nanopore has a field-effect transistor (FET) embedded in the nanopore, i.e. becoming a so called FET nanopore sensor also known as nanopore FET (NPFET). A field-effect transistor is a type of transistor that uses an electric field to control the flow of current in a semiconductor. FETs are devices with three terminals: source, gate, and drain. FETs control the flow of current by the application of a voltage to the gate, which in turn alters the conductivity between the drain and source. The most common type of FET is a MOSFET (metal-oxide-semiconductor field-effect transistor), which utilizes an insulator (typically SiO2) between the gate and the body. Barcode readout with nanopore FET arrays are very effective, with superior throughput, dynamic range, and multiplexing ability. For multiplexing, longer bar codes create billions of labels while e.g. fluorescent labels are limited to 16. For throughput, nanopore arrays with throughputs up to 1 million molecules/s per pore may be attained, giving rise to a bandwidth advantage (>1 MHz) of NPFET. For dynamic range, it may pick up the least abundant species, offering high throughput and single molecule sensitivity.

The nanopore FET of the present disclosure should have a high bandwidth, such as 1 MHz or higher. To provide a high SNR at high bandwidth for the short identification molecules, the aperture of the nanopore FET should have specified dimensions. The aperture should have a height of about 3.4-14 nm, approximately corresponding to a resolution of modifications spaced 10-40 nt apart on the identification molecule. Preferably, the height of the aperture is less than 10 nm, i.e. 3.4-10 nm. The diameter of the aperture should also be controlled, and preferably be in the range of 1-20 nm, such as 3-10 nm, preferably less than 10 nm.

An illustration of a cross-section of a nanopore FET device is shown in FIG. 9. The aperture (1) is here defined as the narrowest constriction of the nanopore device. The aperture has nanoscale dimensions. Its diameter (2) and height (3) most strongly determine the strength of the signal generated when a molecule translocates the nanopore device. The aperture also determines the resolution of the nanopore device, which is here defined as the shortest interlabel distance at which the device can still discriminate the different labels on a double stranded DNA backbone.

Thus, the present disclosure provides an identification molecule for use in nanopore sensing, wherein the identification molecule is able to generate a unique identification signal when translocated through a nanopore sensor, the identification molecule comprising a backbone molecule having a plurality of backbone modifications, wherein each backbone modification give rise to a signal magnitude modulation when translocated through the nanopore sensor, and wherein the distance between the backbone modifications of the identification molecule is between 3.4-13.6 nm, corresponding to a distance of 10-40 nucleotides (nt) or base pairs (bp) (where the average nt length is 0.34 nm).

The spacing between the backbone modifications on the backbone molecule of the identification molecule should thus be dense, being 10-40 nucleotides apart in a DNA backbone. In preferred embodiments, the modifications are spaced less than 10 nm apart, such as 6.8 nm, 5.1, nm or 3.4 nm, corresponding to a distance of 30 nt, 20 nt, 15 nt or 10 nt, such as preferably 10 or 15 nt apart.

The length of the identification molecules of the present disclosure are much shorter than in the prior art, which is enabled by using closely spaced modifications. The length of the identification molecule, or backbone thereof, in the present disclosure is in the range of 6.8-136 nm, corresponding to 20-400 nt (where the average nt length is 0.34 nm). Preferably, the length is less than 102 nm (corresponding to 300 nt), and even more preferably less than 68 nm (corresponding to 200 nt). The length may be for example 34 nm, 27.2nm, 20.4 nm, 13.6 nm, or 6.8 nm, corresponding to 100nt, 80 nt, 60 nt, 40 nt or 20 nt. If the length of the molecule is in the lower range, the modification distance is also in the lower range, such as 10 nt apart, such that the length of the backbone is always larger than the distance between the backbone modifications.

In the present identification molecule, the backbone modifications are selected from a plurality of modification types, and, upon readout in the nanopore sensor, each signal magnitude modulation attained from of the plurality of modifications is assigned an identification value, and wherein all the identification values of the corresponding modifications give rise to an array of identification values corresponding to the unique identification signal of the identification molecule. The identification values are assigned based on i) a distance between the modifications, or ii) the modification type.

In some embodiments, two modification types are used, and each signal magnitude modulation of the two modification types is assigned an identification value of “0” or “1”, and all the identification values of the corresponding modifications give rise to an array of binary identification values corresponding to the unique identification signal of the identification molecule.

In preferred embodiments, the identification molecule has a biopolymer backbone, such as a DNA molecule backbone, preferably a double stranded DNA molecule. The backbone modifications are selected from bulky DNA structures, such as DNA hairpin structures, cruciform DNA, DNA-origami, and quadruplex DNA, DNA-modifications, such as biotin, oligo, peptide, PNA, saccharide, or organic molecules, and differentiated DNA structures, such as ssDNA differentiated into ssDNA hybridized with oligos In some embodiments, the backbone is encoded as a chain structure of blocks formed from one or more layers of a nucleic acid, peptide, or protein.

In preferred embodiments, the nanopore sensor has a field effect transistor (FET) embedded in the nanopore.

According to the present disclosure is also provided methods for using the present identification molecules in assays. For example, methods for detecting or quantifying a target molecule using the present identification molecules is presented. In one example, a method for detecting or quantifying one or more target molecules in a sample using a nanopore sensor comprises providing (S1) a sample comprising one or more target molecules to be detected/quantified, providing (S2) at least one (short) identification molecule as described above and below for the respective target molecules, wherein the identification molecule is able to generate a unique identification signal when translocated through a nanopore sensor and where a correlation between the unique identification signal of the identification molecule and the bound target molecule is known, incubating (S4) the at least one identification molecule with a sample potentially comprising one or more target molecules, thereby allowing the at least one identification molecule to bind to the one or more target molecules forming at least one target-identification molecule, or alternatively, linking the identification molecule to an assay molecule, and allowing the assay molecule to bind the target molecule, forming one or more target-assay-identification molecule, translocating (S5) the target-identification molecule (or alternatively target-assay-identification molecule or a separated identification molecule only) through a nanopore sensor to obtain at least one unique identification signal, and detecting (S6) any presence of the one or more target molecules in a sample by correlating the obtained at least one unique identification signal to the one or more target molecules. Preferably, the nanopore sensor has a field effect transistor (FET) embedded in the nanopore.

Further is provided a system for target molecule sensing and sequencing using a nanopore sensor, the system comprising: a first electrolyte reservoir and a second electrolyte reservoir, the first and second reservoirs being separated by a barrier comprising one or more FET embedded nanopore comprising an aperture of a predetermined height and a predetermined diameter, and optionally, electrodes for translocating molecules through the nanopore from the first electrolyte reservoir to the second electrolyte reservoir, wherein at least one of the first and second electrolyte reservoirs comprises identification molecules of the present disclosure, wherein the identification molecules are able to generate a unique identification signal when translocated through the nanopore.

The aperture is the opening at the bottom of the nanopore channel where the identification molecule is read. A narrow diameter and low height of said aperture give rise to a strong signal when reading the identification molecule.

In an embodiment, the aperture height h of the FET embedded nanopore is predetermined based on the distance between the backbone modifications of the identification molecule, such that a shorter distance between the backbone modifications determines a shorter aperture height. The aperture height may be in the range of 3.4-14 nm, preferably 7 nm, or less. This corresponds to a spacing between the modifications of 10-40 nt, preferably 20 nt or less.

the aperture diameter is in the range of 3-20 nm, preferably 10 nm, or less. The nanopore FET preferably has a bandwidth of 1 MHz or higher.

The system may be used for carrying out a method of detecting one or more target molecules in a sample using a nanopore sensor, the method comprising: providing (S1) a sample comprising one or more target molecules to be detected; providing (S2) at least one identification molecule according to the present disclosure for the respective target molecules, wherein the identification molecule is able to generate a unique identification signal when translocated through a nanopore sensor and a correlation between the unique identification signal of the identification molecule and the bound target molecule is known; optionally linking (S3) the at least one identification molecule to an assay molecule; incubating (S4) the at least one identification molecule with a sample potentially comprising one or more target molecules, thereby allowing the at least one identification molecule to bind to the one or more target molecules forming at least one target-identification molecule or target-assay-identification molecule, translocating (S5) the identification molecule through a nanopore sensor to obtain at least one unique identification signal, wherein the identification molecule is in a complex or optionally separated from the target-identification molecule or target-assay-identification molecule; and detecting (S6) any presence of the one or more target molecules in a sample by correlating the obtained at least one unique identification signal to the one or more target molecules.

In the drawings and specification, there have been disclosed exemplary aspects of the disclosure. However, many variations and modifications can be made to these aspects without substantially departing from the principles of the present disclosure. Thus, the disclosure should be regarded as illustrative rather than restrictive, and not as being limited to the particular aspects discussed above. Accordingly, although specific terms are employed, they are used in a generic and descriptive sense only and not for purposes of limitation.

The description of the example embodiments provided herein have been presented for purposes of illustration. The description is not intended to be exhaustive or to limit example embodiments to the precise form disclosed, and modifications and variations are possible in light of the above teachings or may be acquired from practice of various alternatives to the provided embodiments. The examples discussed herein were chosen and described in order to explain the principles and the nature of various example embodiments and its practical application to enable one skilled in the art to utilize the example embodiments in various manners and with various modifications as are suited to the particular use contemplated. The features of the embodiments described herein may be combined in all possible combinations of methods, products, and systems. It should be appreciated that the example embodiments presented herein may be practiced in any combination with each other. The inventive concept has mainly been described above with reference to a few embodiments, however, as is readily appreciated by a person skilled in the art, other embodiments than the ones disclosed above are equally possible within the scope of the inventive concept, as defined by the appended patent claims.

It should be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be realized in the broadest sense of the claims. All cited references are incorporated by reference in the extent allowed.

1. An identification molecule for use in nanopore sensing, wherein the identification molecule is able to generate a unique identification signal when translocated through a nanopore sensor, the identification molecule comprising a backbone molecule having a plurality of backbone modifications, wherein each backbone modification give rise to a signal magnitude modulation when translocated through the nanopore sensor, and wherein the distance between the backbone modifications of the identification molecule is between 3.4-13.6 nm, corresponding to a distance of 10-40 nucleotides (nt).

2. The identification molecule according to claim 1, wherein the length of the backbone molecule is between 6.8-136 nm, corresponding to 20-400 nt, wherein the length of the backbone molecule is larger than the distance between the backbone modifications.

3. The identification molecule according to claim 1, wherein the distance between the backbone modifications of the identification molecule is less than 10 nm, such as 6.8 nm, 5.1 nm or 3.4 nm.

4. The identification molecule according to claim 2, wherein the length of the backbone molecule is 102 nm, or less, such as 68 nm, 34 nm, 27.2 nm, 20.4 nm, 13.6 nm or 6.8 nm.

5. The identification molecule according to claim 1 wherein the backbone modifications are selected from a plurality of modification types, and wherein, upon readout in the nanopore sensor, each signal magnitude modulation attained from the plurality of modifications is assigned an identification value, and wherein all the identification values of the corresponding modifications give rise to an array of identification values corresponding to the unique identification signal of the identification molecule.

6. The identification molecule according to claim 5, wherein the identification values are assigned based on i) a distance between the modifications, or ii) the modification type.

7. The identification molecule according to claim 1, wherein two modification types are used, and each signal magnitude modulation of the two modification types is assigned an identification value of “0” or “1”, and wherein all the identification values of the corresponding modifications give rise to an array of binary identification values corresponding to the unique identification signal of the identification molecule.

8. The identification molecule according to any one of claim 1, wherein the backbone molecule is a biopolymer backbone, such as a double stranded DNA molecule, or wherein the backbone is encoded as a chain structure of block formed from one or more layers of a nucleic acid, peptide, peptide nucleic acid, or protein.

9. The identification molecule of claim 1, wherein the one or more backbone modifications are selected from bulky DNA structures, such as DNA hairpin structures, cruciform DNA, DNA-origami, and quadruplex DNA, DNA-modifications, such as biotin, oligo, peptide, PNA, saccharide, or organic molecules, and differentiated DNA structures, such as ssDNA differentiated into ssDNA hybridized with oligos”, or a combination thereof.

10. The identification molecule according to claim 1, wherein the nanopore sensor has a field effect transistor (FET) embedded in the nanopore or comprises a remote extended FET, where an electrode wrapped around the nanopore is connected to a remote gate sensor.

11. A system for target molecule sensing and sequencing using a nanopore sensor, the system comprising:
a first electrolyte reservoir and a second electrolyte reservoir, the first and second reservoirs being separated by a barrier comprising one or more FET embedded nanopore comprising an aperture of a predetermined height and diameter; and
optionally, electrodes for translocating molecules through the nanopore from the first electrolyte reservoir to the second electrolyte reservoir,
wherein at least one of the first and second electrolyte reservoirs comprises identification molecules, according to claim 1 wherein the identification molecules are able to generate a unique identification signal when translocated through the nanopore.

12. The system according to claim 11, wherein the aperture height is in the range of 3.4-14 nm, preferably 7 nm, or less.

13. The system according to claim 11, wherein the aperture diameter is in the range of 3-20 nm, preferably 10 nm, or less.

14. The system according to claim 11, wherein the bandwidth of the FET embedded nanopore is at least 1 MHz.

15. A method for detecting one or more target molecules in a sample using a nanopore sensor, the method comprising:
providing a sample comprising one or more target molecules to be detected;
providing at least one identification molecule according to claim 1 for the respective target molecules, wherein the identification molecule is able to generate a unique identification signal when translocated through a nanopore sensor and a correlation between the unique identification signal of the identification molecule and the bound target molecule is known;
optionally linking the at least one identification molecule to an assay molecule;
incubating the at least one identification molecule with a sample potentially comprising one or more target molecules, thereby allowing the at least one identification molecule to bind to the one or more target molecules forming at least one target-identification molecule or target-assay-identification molecule,
translocating the identification molecule through a nanopore sensor to obtain at least one unique identification signal, wherein the identification molecule is in a complex or optionally separated from the target-identification molecule or target-assay-identification molecule; and
detecting any presence of the one or more target molecules in a sample by correlating the obtained at least one unique identification signal to the one or more target molecules.
